(19) United States
(12) Patent Application Publication
(10) Pub. No.: US 2026/0295247 A1
(43) Pub. Date: Oct. 1, 2026

(54) MALE ORGASM ENHANCEMENT DEVICE AND METHOD

(71) Applicant: VIRILITY MEDICAL LTD., HOD-HASHARON (IL)

(72) Inventors: Tal GOLLAN, Ramat-Gan (IL); Efrat RIKLIN, Hod-HaSharon (IL); Nir TSOURI, Hod-HaSharon (IL); David SALTON, Hod Hasharon (IL)

(21) Appl. No.: 19/489,349

(22) PCT Filed: Jun. 11, 2024
(86) PCT No.: PCT/IL2024/050580

§ 371 Date: Dec. 2, 2025

Related U.S. Application Data
(60) Provisional application No. 63/507,473, filed on Jun. 12, 2023.

(51) Int. Cl. A61N 1/36; A61N 1/04

(52) U.S. Cl. CPC A61N 1/36007; A61N 1/0452; A61N 1/0456; A61N 1/0492; A61N 1/36003; A61N 1/36034

(57) ABSTRACT

In some embodiments, the present invention provides a device, wherein the device comprises a muscle stimulator, configured to attach to the skin surface of the perineum and other skin surfaces of a subject wishing to enhance orgasm, wherein the muscle stimulator contains electrodes configured to deliver electrical or magnetic impulses transcutaneously to the bulbocavernosus muscle and/or seminal vesicles and/or prostate of the subject, wherein the transcutaneously delivered impulses are configured to enhance and/or prolong male orgasm.

### RELATED APPLICATIONS

This international application claims the priority benefit of U.S. provisional Application no. 63/507,473, incorporated herein by reference in its entirety.

The following international applications are incorporated herein by reference in their entirety:
PCT/IB2016/001731 “Transcutaneous Electrical Muscle Stimulation Device for the Treatment of Premature Ejaculation or Erectile Dysfunction, and Methods of Use Thereof”
PCT/IL2018/051402 “Ejaculation Control”

### TECHNICAL FIELD

Various embodiments of the present invention relate to devices and methods to enhance orgasm. In particular, various embodiments of the present invention relate to devices and methods to enhance or prolong male orgasm.

### BACKGROUND

The male orgasm, the climax of sexual excitation, often includes neural, vascular, and hormonal responses. While male ejaculation is a peripheral and spinal event, orgasm is a supraspinal event. The present invention describes a device and method to enhance and/or prolong the male orgasm, by increasing peripheral excitation.

Aydin et al¹ describes the presence of taste bud-like structures (TBLS) in the penile urethra and hypothesized that TBLS are stimulated during ejaculation by fructose, which is secreted by the seminal vesicles and is part of the semen. TBLS stimulation by fructose therefore may be a contributor to orgasmic sensation.

Medved et al² reported of significant change in seminal vesicle volume after ejaculation but found no significant change in volume of the prostate. Considering that seminal fructose is secreted by the seminal vesicles³, it can therefore be hypothesized that stimulation of the smooth muscles of the seminal vesicles can enhance and prolong the male orgasm. This is contrary to common belief that the prostate is a major contributor to orgasmic sensation, and occasionally referred to as the ‘male G-spot.’

### SUMMARY OF THE INVENTION

In some embodiments, the present invention provides device, method and clinical results, wherein the device comprises a nerve or muscle stimulator, configured to attach to the skin surface of the perineum and other surfaces of a subject who wishes to enhance his orgasm sensation, wherein the nerve or muscle stimulator contains electrodes configured to deliver transcutaneous electrical or magnetic impulses to the pelvis of the subject, wherein the transcutaneously delivered impulses are configured to enhance and/or prolong orgasmic sensation.

In some embodiments, the electrical or magnetic transcutaneous impulses are delivered to the neuromuscular junction of the nerve innervating the bulbocavernosus muscle and/or the nerves innervating the prostate and/or the nerves innervating the seminal vesicles. In some embodiments, the nerve innervating the bulbocavernosus muscle is the motor branch of the pudendal nerve; and/or the nerve innervating the prostate is one or more of the pelvic nerves (such as the pelvic splanchnic nerve and/or the sacral nerves) and one or more of the hypogastric nerves (such as sympathetic fibers descending from the superior hypogastric plexus, and/or preganglionic parasympathetic fibers originating from the pelvic splanchnic nerves, and/or visceral sensory fibers that project to the lumbar spinal cord); and/or the nerve innervating the seminal vesicles is the hypogastric nerves and the inferior hypogastric plexus.

In some embodiments, the present invention provides a device or skin patch, wherein the device comprises a nerve or muscle stimulator, configured to attach to the skin surface of the perineum and other surfaces, such as abdomen or lower back, of a subject who wishes to enhance and/or prolong his orgasm, wherein the skin patch contains electrodes configured to deliver electrical or magnetic impulses transcutaneously to the bulbospongiosus muscles and/or smooth muscles of the prostate and/or smooth muscles of the seminal vesicles of the subject, and wherein the transcutaneously delivered impulses are configured to enhance and/or prolong orgasm.

In some embodiments, the device comprises at least one electrode applied to the perineal skin, and another electrode, applied to the perineal skin, and/or another electrode, applied to the abdomen skin, and/or another electrode, applied to the skin of the lower back.

In some embodiments, the device has two electrodes.

In some embodiments, the device has three or four electrodes.

In some embodiments, muscle simulator pad 110 is configured to be entirely in contact with the perineum of the subject.

In some embodiments, the transcutaneously delivered electrical or magnetic impulses are configured to enhance and/or prolong male orgasm.

In some embodiments, the transcutaneously delivered electrical or magnetic impulses are configured to induce a continuous contraction of the bulbocavernosus muscle 120 of the subject.

In some embodiments, the transcutaneously delivered electrical or magnetic impulses are configured to enhance male orgasm, by increasing the contractility of the smooth muscles surrounding the prostate 125 and/or the seminal vesicles 130.

In some embodiments, the transcutaneously delivered electrical or magnetic impulses are configured to enhance male orgasm, by increasing the fluid volume secreted by the prostate 125 and/or seminal vesicles 130.

In some embodiments, the device 100 is disposable.

In some embodiments, the device 100 allows repetitive use.

In some embodiments, the present invention provides a method, wherein the method treats a subject who wishes to enhance male orgasm, comprising the steps of: (a) attaching the device according to some embodiments of the present invention to the skin surface of the perineum of the subject; and (b) attaching additional electrodes to the skin surface of one or more of the perineum and/or abdomen and/or lower back; and (c) delivering electrical or magnetic impulses transcutaneously to the bulbocavernosus muscle and/or to the prostate and/or to the seminal vesicles of the subject, wherein the transcutaneously delivered electrical or magnetic impulses are configured to enhance and/or prolong orgasm.

In some embodiments, the device is attached to the skin surfaces of the subject prior to sexual intercourse.

In some embodiments, the impulses are delivered transcutaneously to the pelvis of the subject until the subject orgasms and/or ejaculates.

In some embodiments, the impulses are delivered transcutaneously to the pelvis of the subject until the subject wishes to orgasm and/or ejaculate.

In some embodiments, the frequency of the electrical impulses is from 0.5 to 200 Hz.

In some embodiments, the pulse width of an electrical impulse is from 15 to 800 μs.

In some embodiments, the interphase interval is from 10 to 250 μs.

In some embodiments, the current of the electrical impulses is from 0.5 to 80 mA.

In some embodiments, the electrical impulses induce a continuous contraction of the bulbocavernosus muscle of the subject. The continuous contraction may be tetanic, sub-tetanic, a sub-tetanic contraction with smaller oscillations, continuous, and/or intermittent.

In some embodiments, the impulses increase the contractility of the bulbospongiosus muscles and/or the smooth muscles surrounding the prostate and/or seminal vesicles of the subject.

In some embodiments, the present invention provides a device for enhancement of a male orgasm, comprising
a plurality of muscle stimulation pads configured to attach to a perineum of a subject and at least one of
   an abdomen of the subject; and
   a lower back of the subject;
the muscle stimulation pads each comprising one or more electrodes configured to deliver transcutaneous electrical or magnetic impulses, wherein
the transcutaneous impulses are delivered to one or more of nerves innervating the seminal vesicles; and nerves innervating the prostate; and
the transcutaneously delivered impulses are configured to intensify, enhance, and/or prolong orgasmic sensation.

In some embodiments, the device is further configured to deliver the transcutaneous impulses a neuromuscular junction of a nerve innervating the bulbocavernosus muscle.

In some embodiments, wherein the nerves innervating the seminal vesicles comprise hypogastric nerves and an inferior hypogastric plexus.

In some embodiments, wherein the nerves innervating the prostate comprise pelvic nerves and hypogastric nerves.

In some embodiments, wherein the nerve innervating the bulbocavernosus muscle comprises a motor branch of the pudendal nerve.

In some embodiments, comprising two electrodes.

In some embodiments, comprising three electrodes.

In some embodiments, comprising four electrodes.

In some embodiments, one of the pads is configured to be entirely in contact with the perinium of the subject.

In some embodiments, the transcutaneously delivered electrical or magnetic impulses are configured to induce a continuous contraction of the bulbocavernosus muscle of the subject.

In some embodiments, the transcutaneously delivered electrical or magnetic impulses are configured to enhance the male orgasm by increasing contractility of the smooth muscles surrounding at least one of the prostate and the seminal vesicles.

In some embodiments, the transcutaneously delivered electrical or magnetic impulses are configured enhance the male orgasm by increasing the fluid volume secreted by the prostate and/or seminal vesicles.

In some embodiments, wherein the device is disposable.

In some embodiments, the device allows repetitive use.

In some embodiments, a voltage of the transcutaneously delivered impulses is between about 1 and 125 volts.

In some embodiments, a current of the transcutaneously delivered impulses is between about 0.5 and 70 milliamperes.

In some embodiments, wherein a pulse width of the transcutaneously delivered impulses is between about 15 and 250 microseconds.

In some embodiments, a repetition rate of the transcutaneously delivered impulses is between about 0.5 and 200 pulses per second.

In some embodiments, the transcutaneously delivered impulses generate increased intraurethral pressure, and/or stronger bulbospongiosus muscle contractions of the subject.

In some embodiments, the transcutaneously delivered impulses generate greater fructose availability in tastebud-like structures in a urethra of the subject.

In some embodiments, the transcutaneously delivered impulses generate stronger neural signaling to higher neural centers.

In some embodiments, the transcutaneously delivered impulses induce a more intense orgasm sensation for the subject.

In some embodiments, further configured for calibration comprising setting an intensity of the impulses to a muscle contraction threshold intensity.

In some embodiments, the present invention provides a device for enhancement of a male orgasm, comprising
two or more muscle stimulation pads configured to attach to two or more of the following skin surfaces of a subject:
   a perineum;
   an abdomen; and
   a lower back
one or more electrodes, in each of the muscle stimulation pads, configured to deliver transcutaneous electrical or magnetic impulses, wherein the transcutaneous impulses are delivered to one or more of
   a neuromuscular junction of a nerve innervating the bulbocavernosus muscle;
   nerves innervating the prostate; and
   nerves innervating the seminal vesicles; and
the transcutaneously delivered impulses are configured to intensify, enhance, and/or prolong orgasmic sensation.

In some embodiments, the present invention provides a method for enhancement of a male orgasm, comprising
attaching a plurality of muscle stimulation pads of the device of claim 1 to a perineum of a subject and at least one of
   an abdomen of the subject; and
   a lower back of the subject;
delivering electrical or magnetic impulses, by the device, to one or more of
   nerves innervating the prostate; and
   nerves innervating the seminal vesicles, wherein
the transcutaneously delivered impulses are configured to intensify, enhance, and/or prolong orgasmic sensation.

In some embodiments, comprising attaching a muscle simulation pad to one or more of an abdomen and a lower back of the subject.

In some embodiments, the nerve innervating the bulbocavernosus muscle comprises a motor branch of the pudendal nerve.

In some embodiments, the nerves innervating the prostate comprise pelvic nerves and hypogastric nerves.

In some embodiments, the nerves innervating the seminal vesicles comprise hypogastric nerves and an inferior hypogastric plexus.

In some embodiments, further comprising the muscle stimulation pad being entirely in contact with the perinium of the subject by.

In some embodiments, further comprising inducing a continuous contraction of the bulbocavernosus muscle of the subject, by the transcutaneously delivered electrical or magnetic impulses.

In some embodiments, further comprising enhancing the male orgasm by increasing contractility of the smooth muscles surrounding at least one of the prostate and the seminal vesicles, by the transcutaneously delivered electrical or magnetic impulses.

In some embodiments, further comprising enhancing the male orgasm by increasing the fluid volume secreted by the prostate and/or seminal vesicles, by the transcutaneously delivered electrical or magnetic impulses.

In some embodiments, further comprising disposal of the muscle simulation pad(s) after a single use.

In some embodiments, further comprising repetitively using the muscle simulation pad(s).

In some embodiments, further comprising generating increased intraurethral pressure and/or stronger bulbospongiosus muscle contractions of the subject by the transcutaneously delivered impulses.

In some embodiments, further comprising generating greater fructose availability in tastebud-like structures in a urethra of the subject by the transcutaneously delivered impulses.

In some embodiments, further comprising generating stronger neural signaling to higher neural centers.

In some embodiments, further comprising inducing a more intense orgasm sensation for the subject by the transcutaneously delivered impulses.

In some embodiments, further comprising calibrating a muscle simulator pad by increasing an intensity of the electrical or magnetic impulses until reaching a motor threshold of the subject.

In some embodiments, the motor threshold intensity is applied during the delivery of electrical or magnetic impulses.

In some embodiments, the impulses from the muscle simulation pads are delivered simultaneously.

In some embodiments, the impulses from the muscle simulation pads are delivered sequentially. The method of claim 25, wherein the impulses from the muscle simulation pads are delivered in random sequence.

In some embodiments, a sequence and/or electric parameters of the impulses are controlled by the subject from a remote device.

In some embodiments, further comprising calibrating an intensity setting of the impulses to a muscle contraction threshold intensity.

In some embodiments, the present invention provides a device for enhancement of a male orgasm, comprising
muscle stimulation pads configured to attach to the following skin surfaces of
   a subject:
   an abdomen; and
   a lower back
one or more electrodes, in each of the muscle stimulation pads, configured to deliver transcutaneous electrical or magnetic impulses, wherein
   the transcutaneous impulses are delivered to one or more of
      a neuromuscular junction of a nerve innervating the bulbocavernosus muscle;
      nerves innervating the prostate; and
      nerves innervating the seminal vesicles; and
   the transcutaneously delivered impulses are configured to intensify, enhance, and/or prolong orgasmic sensation.

### BRIEF DESCRIPTION OF DRAWINGS

FIG. 1 depicts a device of the present invention attached to the pelvis of a subject, according to some embodiments of the present invention.

FIG. 2 depicts a functional block diagram of a muscle stimulation pad of a device of the present invention, according to some embodiments.

FIG. 3 shows an Orgasmometer⁴ visual analogue scale used by subjects in a clinical study to report orgasm intensity, according to some embodiments.

FOG. 4A depicts a method of use of the device, according to some embodiments.

FIG. 4B depicts a method of calibration of the device, according to some embodiments.

FIG. 5 depicts clinical results reported by subjects who used a stimulation device, according to some embodiments.

FIG. 6 depicts a flowchart of physiological events to enhance and/or prolong orgasm, according to some embodiments.

### DESCRIPTION OF EXEMPLARY EMBODIMENTS

### The Device

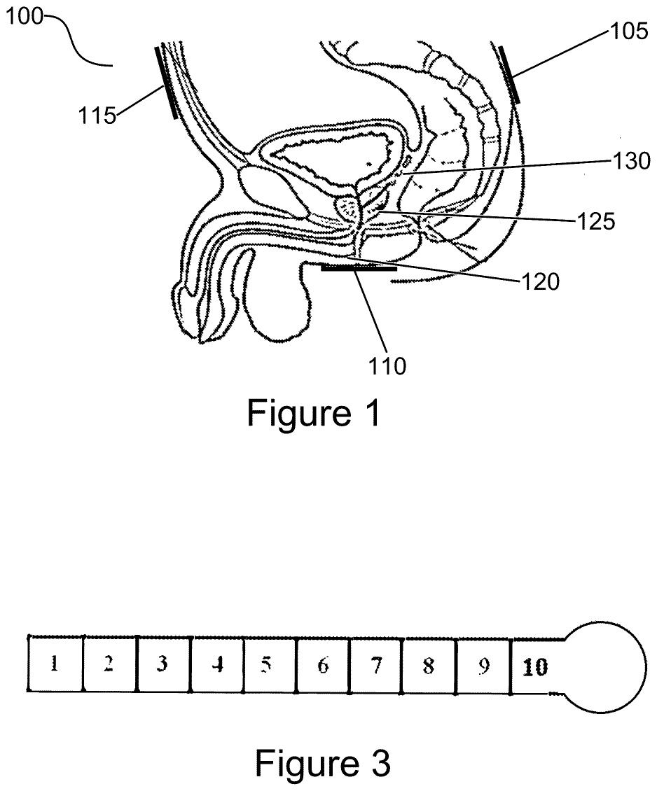

Referring to FIG. 1, in some embodiments, the present invention provides a device 100, wherein the device 100 comprises one or more nerve or muscle stimulator pads 105, 110, 115, configured to be attached to skin surfaces of a subject who wishes to enhance orgasm. In some embodiments, a muscle stimulator pad 110 is configured to attach to the skin surface of the perineum. In some embodiments, a muscle stimulator pad 115 is configured to attach to the skin surface of the abdomen. In some embodiments, a muscle stimulator pad 105 is configured to be attached to the lower back. The device 100 contains electrodes—disposed among the muscle stimulator pads 105, 110, 115—configured to deliver electrical or magnetic impulses transcutaneously to the pelvic organs.

In some embodiments, the impulses are delivered transcutaneously to the neuromuscular junction of the nerve innervating the bulbocavernosus muscle 120.

In some embodiments, the nerve innervating the bulbocavernosus muscle 120 is the motor branch of the pudendal nerve.

In some embodiments, the impulses are delivered transcutaneously to the smooth muscles surrounding the prostate 125.

In some embodiments, the impulses are delivered transcutaneously to the smooth muscles surrounding the seminal vesicles 130.

In some embodiments, the nerve innervating the bulbocavernosus muscle 120 is the motor branch of the pudendal nerve.

In some embodiments, the nerves innervating the prostate 125 are the pelvic nerves and hypogastric nerves.

In some embodiments, the nerves innervating the seminal vesicles 130 are the hypogastric nerves and the inferior hypogastric plexus.

In some embodiments, the device 100 has two electrodes.

In some embodiments, the device 100 has three electrodes.

In some embodiments, the device 100 has four electrodes.

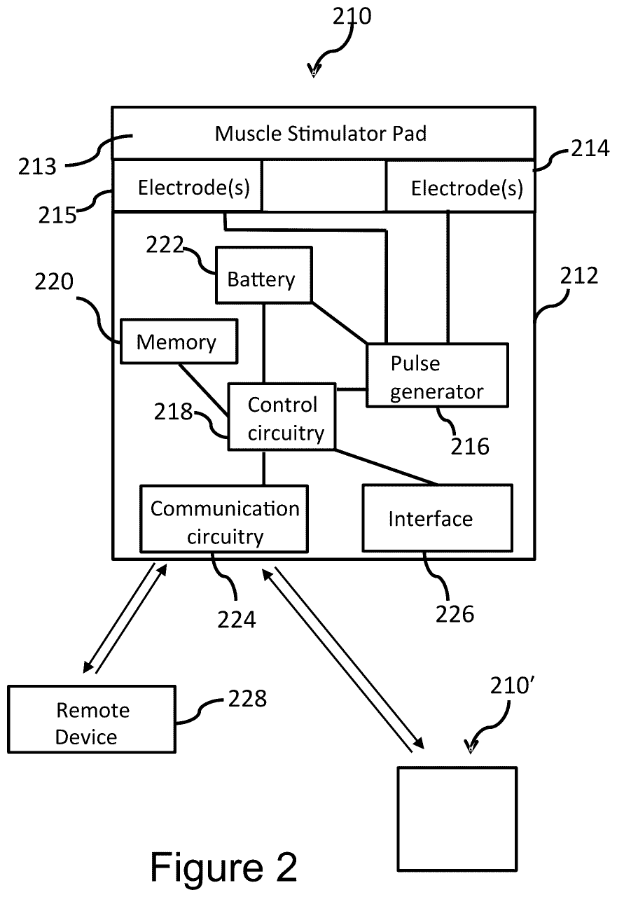

Reference is now made to FIG. 2, depicting the components of a muscle stimulator pad 210 of a device 100, according to some exemplary embodiments of the invention. In some embodiments, the device 100 comprises at least one other muscle stimulator pad 210′.

According to some exemplary embodiments, a muscle stimulator pad 210 comprises a thin housing 212 having an upper flat face and a lower face. In some embodiments, the width of the housing is between 1 mm and 10 mm, for example 1 mm, 2 mm, 3 mm, 4 mm, 5 mm, 7 mm, 9 mm, 10 mm or any intermediate width.

In some embodiments, a housing 212 is shaped and sized to be positioned entirely between the posterior aspect of the scrotum and the anus. In some embodiments, the axial length of the housing 212 is shorter than the anogenital distance.

In some embodiments, the housing 212 comprises axial bending lines, for example to direct the bending of the housing 212 to conform to the anatomical curvature of the skin surface. In some embodiments, the housing 212 comprises an attachment element 213 for attaching the upper flat face of the housing 212 to the skin surface. In some embodiments, the attachment element comprises a sticker with glue, optionally a reusable sticker which allows for example to remove and re-attach the device to the skin surface several times. In some embodiments, the attachment element 213, optionally at the interface between the housing 212 and the skin, comprises a conductive and optionally an adhesive hydrogel, for example to allow better conductance when attaching the device to a hairy skin surface.

According to some exemplary embodiments, each muscle stimulator pad 210 comprises one or more electrodes, for example electrode 214 and, optionally, electrode 215, positioned at least partly on the upper face of the housing 212. Alternatively, the one or more electrodes is/are positioned inside the housing 212 and deliver the electric field through a conductive layer positioned on top of the housing 212.

In some embodiments, the one or more electrodes 214, (215) are positioned along the anogenital distance and/or in parallel to each other.

In some embodiments, at least some of the one or more electrodes 214, (215) are unipolar. In some embodiments, an electric field is established between electrodes 214, (215) in different muscle stimulator pads 210, 210′.

Optionally, some of the electrodes 214, (215) are bipolar. In some embodiments, the electrodes comprise at least one sensing electrode positioned at the upper face of the housing, for example, to measure at least one physiological parameter of the body, for example heart rate and/or electrical conductivity of one or more muscles. In some embodiments, the electrodes, for example electrode 214 and/or electrode 215, have a surface area in a range between 90 mm² and 850 mm² , for example 100 mm² , 200 mm² , 300 mm², 400 mm² or any intermediate, larger or smaller value. In some embodiments, using electrodes with a surface area smaller than 90 mm² may yield large current and power density which may cause pain and discomfort to the user. In some embodiments, using electrodes with surface area larger than 850 mm² may result in large current distribution and inefficient stimulation. According to some exemplary embodiments, the electrodes for example electrodes 214 and electrodes 215 are positioned at a distance of at least 8 mm between each other, for example 10 mm, 11 mm, 12 mm, 13 mm, 14 mm or any intermediate or larger distance.

According to some exemplary embodiments, a muscle stimulator pad 210 comprises a pulse generator positioned inside the housing 212. In some embodiments, the pulse generator 216 is electrically connected to at least some of the electrodes 214. In some embodiments, a control circuitry, for example control circuitry 218 is electrically connected to the pulse generator 216. In some embodiments, the control circuitry 218 signals the pulse generator 216 to generate an electric field according to at least one protocol and/or according to electric field parameter values stored in a memory 220, which is optionally a readable and writable memory. In some embodiments, the electric field parameters comprise intensity, voltage, frequency, interphase interval, pulse width and/or ramp time. In some embodiments, one or more of the elements contained within the pad 210 (such as the memory or the control circuitry) may be moved externally of the pad 210, such as positioning them within a remote device 228.

In some embodiments, voltage output can range between 1 and 120 volts.

In some embodiments, current output can range between 0.5 and 70 milliamperes.

In some embodiments, pulse width output can range between 15 and 250 microseconds.

In some embodiments, frequency output can range between 0.5 and 200 pulses per second.

In some embodiments, intensity of ejaculation is increased.

According to some exemplary embodiments, the device comprises an interface 226 in at least one of the muscle stimulator pads 210, for example for receiving input from a user and/or for delivery of indications to the user. In some embodiments, the interface 226 comprises at least one light source, for example a light emitting diode (LED) and/or at least one sound producing element. In some embodiments, the interface 226 delivers indications related to the treatment protocol and/or the electric field. Alternatively or additionally, the interface 226 delivers indications related to the status of the device 210, for example statuses that the device 100 and/or muscle stimulator pad 210 is on, is delivering an electric field, and/or is in a non-stimulating mode. In some embodiments, the interface 226 delivers alerts to a user, for example a low battery alert and/or alerts related to device malfunctioning.

According to some exemplary embodiments, the muscle stimulator pad 210 comprises a communication circuitry 224 electrically connected to the control circuitry 218 inside the housing 212. In some embodiments, the communication circuitry receives and/or transmits wireless signals, for example Bluetooth signals, WiFi or any other wireless signals. In some embodiments, the communication circuitry 224 comprises a receiver and/or a transmitter.

In some embodiments, the communication circuitry 224 communicates with another muscle stimulator pad 210′, for coordinating operation of the device 100 among each of the muscle stimulator pads 210. In some embodiments, the memory 220 of one of the muscle stimulator pads 210 controls operation of the other muscle simulator pads. For example, the memory 220 of one of a plurality of muscle stimulator pads 210 stores at least one protocol and/or electric field parameter value of another muscle stimulator pad 210′ and transmits same to the other muscle stimulator pad 210′.

In some embodiments, the communication circuitry 224 communicates with a remote device 228, for example a wearable device, a mobile device, and/or a computer. In some embodiments, the remote device 228 controls operation of the muscle simulator pads 210. For example, the remote device 228 stores at least one protocol and/or electric field parameter value of another muscle stimulator pad 210′ and transmits same to the other muscle stimulator pad 210′.

According to some exemplary embodiments, the device 210 comprises at least one battery, for example battery 222 inside the housing 212. In some embodiments the battery 222 is a rechargeable battery, for example a lithium-ion battery. In some embodiments, the battery 222 is remotely charged. Alternatively, the battery 222 is a non-rechargeable battery. Optionally, the battery is a thin battery, for example a coin or a disc shaped battery. In some embodiments, the battery 222 is a replaceable battery, for example a battery that can be replaced by the removal of a cover in the housing 212.

According to some exemplary embodiments, the device 210 measures and/or calculates at least one electrical parameter of the skin, for example, impedance. In some embodiments, the electrical parameter of the skin is measured by at least one electrode or at least one sensor of the device which is in electrical contact with the skin. In some embodiments, monitoring the electrical impedance may be used to determine the quality of adhesion of the device to the skin, prior to the delivery of the electric field and/or during the delivery of the electric field. In some embodiments, high impedance values, for example impedance values of at least 5000 ohm, for example 5000 ohm, 6000 ohm, 7000 ohm or any intermediate or larger value indicate that the device has no contact with the skin.

In some embodiments, in this case, the device would automatically cease stimulation and optionally signal a mobile device, for example a smartphone, to deliver an alert to the user. In some embodiments, gradually decreasing impedance values, for example at a rate of at least 50 ohm per second, for example 50 ohm per second, 100 ohm per second, 500 ohm per second or any intermediate or larger rate may be an indication of nerve activity which may indicate approaching ejaculation and/or urination.

According to some exemplary embodiments, low impedance values, for example, impedance values of 1000 ohm and lower, for example 900 ohm, 800 ohm, 700 ohm or any intermediate or smaller value would indicate that the device is properly contacting the skin. In some embodiments, in this case, the device may signal the smartphone to deliver an indication to the user that the device is properly attached to the skin.

According to some exemplary embodiments, measured impedance values in a range between 1000 ohm and 5000 ohm indicates that the device is sub-optimally applied to the skin. In some embodiments, in this case, the device would signal the smartphone to generate a warning indication to the user.

According to some embodiments, if the measured or calculated impedance values indicates an insufficient contact between electrodes of the device and the perineum surface or perineum skin, the device stops generation of the electric field and/or delivers an indication to a user. Optionally, the indication to the user is delivered by a mobile device wirelessly coupled to the device.

Impulses may be applied between electrodes on the same muscle stimulation pads 210. Alternatively, or additionally, impulses may be applied between electrodes on different muscle stimulation pads 210, 210′.

Referring again to FIG. 1, there are three muscle stimulation pads illustrated, one 110 positioned in the area of the perineum, another positioned in the area of the lower back 105 and another positioned in the area of the lower abdomen 115. The control circuitry 224 may be controlled, by the remote device 118 or be programmed to cause the pulse generator 216 to deliver electrical or magnetic pulses to the pads 110, 105 and 115 either simultaneously or sequentially depending on the stimulation and desired effects. Alternatively, pulses may be delivered to only one of the pads or to only two of the pads, again based upon the desired result. Further alternatively, pulses may be delivered in a random sequence or delivered in a desired sequence and then stopped temporarily before being resumed. Feedback, either directly from the user or as a result of electronic feedback to the remote device, may be used to insert into memory 220 the sequence of stimulations deemed most efficacious by the user.

In some embodiments, the device is optionally usable for training to strengthen pelvic-floor muscles. Such strengthening may further enhance or intensify the sensation of orgasm. Since part of the orgasmic pleasure is from the contractions of the pelvic-floor muscles, strengthening the muscles may help intensify the sensation of orgasm. Such training usage of the device may be in addition to, or instead of, usage during sexual intercourse.

### Methods of Use and Calibration

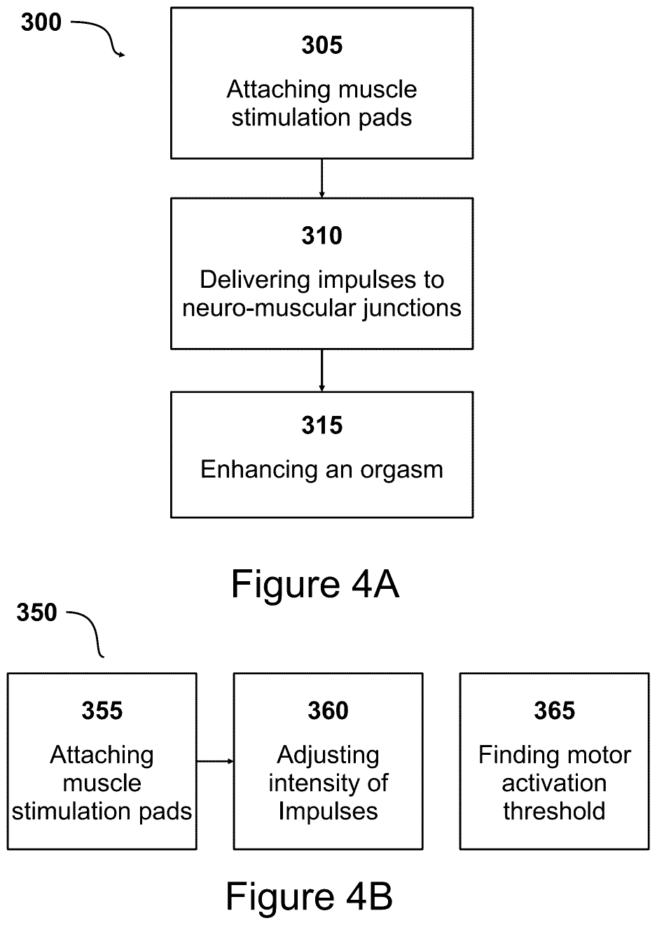

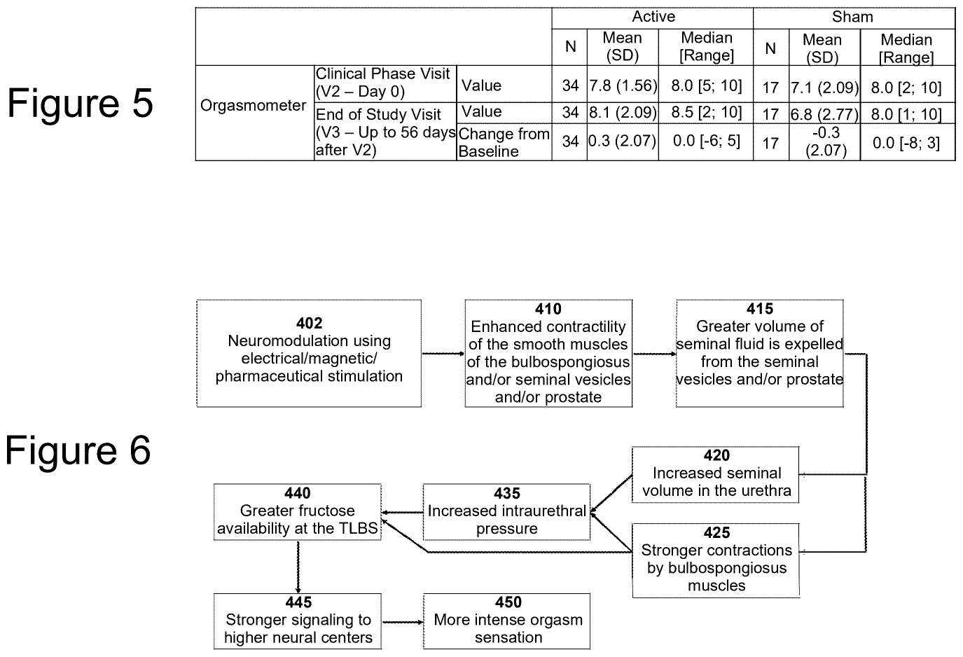

Referring to FIG. 4A, in some embodiments the invention provides a method of use 300 for a device of the present invention, such as one of the embodiments disclosed herein.

The method 300 may comprise attaching muscle stimulation pads of the device to one or more skin surfaces of a subject 305. In some embodiments, a muscle stimulation pad is attached to the perineum and, optionally, one or more of the abdomen and lower back. In some embodiments, a muscle simulation pad is attached to any one or more of the perineum, the abdomen, and the lower back; for example, the perineum and the abdomen, the perineum and the lower back, the lower back and the abdomen, or all three skin surfaces: the perineum, the abdomen, and the lower back.

The method 300 may comprise delivering electrical or magnetic impulses 310, by the muscle simulation pads, to neuro-muscular junctions of the subject. The neuro-muscular junctions may include any one or more of 1) a neuromuscular junction of a nerve innervating the bulbocavernosus muscle; nerves innervating the prostate; and nerves innervating the seminal vesicles. The electrical or magnetic pulses may be delivered by the pads either simultaneously or sequentially depending on the stimulation and desired effect(s). Alternatively, pulses may be delivered by only one of the pads or by only two of the pads, again based upon the desired result. Further alternatively, pulses may be delivered in a random sequence or delivered in a desired sequence and then stopped temporarily before being resumed. Feedback, either directly from the user or as a result of electronic feedback to a remote device, may be used to provide a sequence of stimulations deemed most efficacious by the user.

The method 300 may further comprise enhancing, intensifying, and/or prolonging a male orgasm 315. In addition, or alternatively, intensifying and/or prolonging an ejaculation.

Referring to FIG. 4B, in some embodiments the invention provides a method of calibration 350 for a device of the present invention.

The calibration method 350 may comprise attaching muscle stimulation pads of the device to one or more skin surfaces of a subject 355.

The calibration method 350 may comprise adjusting (e.g. incrementally increasing) intensity of impulses 360 to one or more muscles that are the target of the electrical or magnetic impulses. In some embodiments, intensity adjustment is made from a remote device.

The calibration method 350 may comprise identifying the motor activation threshold intensity 365, which is the impulse intensity at which contraction of desired muscles is achieved. The sensation of muscle contraction may be similar to that induced while temporary and voluntary urination pause. In some embodiments, impulse intensity is reduced if the subject reports a sensation of discomfort or pain.

In some embodiments, calibration is followed by a stimulation protocol. After identifying the subject's electrical stimulation intensity that led to perineal muscle contraction, the subject receives a continuous perineal stimulation (e.g. for a period of 5 minutes) with his perineal contraction intensity (in mA). The intensity is then incrementally increased incrementally (e.g. by 0.5 mA) at the beginning of each successive period or sub-period (e.g. 1 minute), resulting in a final stimulation intensity of a fixed increment (e.g. 2 mA) above the individually measured perineal muscle contraction intensity threshold. In some embodiments, stimulation intensity is limited to 25 mA. Following completion of a stimulation protocol, the subject may undergo a medical examination. In some embodiments, the subject is provided adverse effects follow-up instructions (to be done, for example, 72 hours after the examination) and self-reporting form to take and fill in at home.

In some embodiments, the intensity of impulses during usage 300 is the same as the motor threshold intensity identified during calibration 350.

In some embodiments, calibration 350 is performed by a healthcare professional. In some embodiments, the device is recalibrated, e.g. after a fixed number of uses or if efficacy degrades.

Embodiments of the invention are not limited to this disclosure. A person skilled in the art, having comprehended the teachings of this disclosure, and after reading publications listed in the RELATED APPLICATIONS and REFERENCES CITED, would identify other embodiments based on combinations of disclosures herein and in the other publications. These combinations as well are included in the scope of the present invention.

### Clinical Results

Referring to FIG. 1, in some embodiments, the device generated clinical effect reported by study subjects. The study included two arms; (a) Active arm, where subjects were provided fully functional muscle stimulator for treatment, whereby the device is calibrated to provide impulses at a level of the motor activation threshold intensity; and (b) Sham arm, where subjects were provided sham stimulation, at the level of sensory threshold intensity, incapable of inducing muscle response.

Still referring to FIG. 1, more improvement in orgasmic intensity was reported in the Active group than in the Sham group, comparing before the study (denoted Clinical Phase Visit V2-Day—0) and after the study (denoted End of Study Visit V3).

Referring to FIG. 2, orgasmic intensity was reported using Orgasmometer, a validated patient reported outcome questionnaire for male subjects. The Orgasmometer utilizes visual analogue scale for evaluation of perceived orgasmic intensity.

Referring to FIG. 3, electrodes can be placed in one or more of the perineum, lower back and abdomen.

Still referring to FIG. 3, the targets of delivered transcutaneous stimulation can one or more of the seminal vesicles, prostate and bulbospongiosus muscle.

### Physiological Mechanism of Male Orgasm and its Intensity

Referring to FIG. 6, the physiological events which are required to enhance male orgasm are detailed. Delivery of electrical or magnetic or pharmaceutical stimulation 402 enhances the contractility of the bulbospongiosus muscles and/or the smooth muscles of the seminal vesicles and/or the smooth muscles of the prostate 410.

Still referring to FIG. 6, increased contractility of the smooth muscles of the seminal vesicles and/or the prostate 415, expels great volume of fluid during emission, the first phase of ejaculation.

Still referring to FIG. 6, a great volume of fluid during emission, generates an increase in the volume of semen fluid in the urethra 420 and therefore generates increased intraurethral pressure⁵ 435.

Still referring to FIG. 6, increased intraurethral pressure, and/or stronger bulbospongiosus muscle contractions 425, generate greater fructose availability at the taste bud-like structures in the urethra 440.

Still referring to FIG. 6, greater fructose availability at the taste bud-like structures in the urethra generates stronger neural signaling to higher neural centers 445.

Still referring to FIG. 6, stronger neural signaling to higher neural centers induces a more intense orgasm sensation for the user of the device 450.

### REFERENCES CITED

The following publications are incorporated by reference in their entirety into this disclosure.
¹ Aydin et al., “Taste bud-like structures in penile tissues and a predictive neural mechanism of male orgasm: A preliminary hypothesis based on histological evidence”; Neurology, Psychiatry and Brain Research, 20 (2014) 55-62.
² Medved et al., “MR Imaging of the Prostate and Adjacent Anatomic Structures before, during, and after Ejaculation: Qualitative and Quantitative Evaluation”; Radiology, Volume 271: Number 2—May 2014.
³ Mckay et al., “Anatomy, Abdomen and Pelvis, Seminal Vesicle”; Statpearls, 2021.
⁴ Limoncin et al., “The impact of premature ejaculation on the subjective perception of orgasmic intensity: validation and standardization of the ‘Orgasmometer’”; Andrology, 2016 September;4(5):921-6.
⁵ Shafik, “Response of the Urethral and Intracorporeal Pressures to Cavernosusu Muscle Stimulation: Role of the Muscles in Erection and Ejaculation”; Urology, 46 (1), 1995

1. A device for enhancement of a male orgasm, comprising
a plurality of muscle stimulation pads configured to attach to a perineum of a subject and at least one of
an abdomen of said subject; and
a lower back of said subject;
said muscle stimulation pads each comprising one or more electrodes configured to deliver transcutaneous electrical or magnetic impulses, wherein
said transcutaneous impulses are delivered to one or more of
nerves innervating the seminal vesicles; and
nerves innervating the prostate; and
said transcutaneously delivered impulses are configured to intensify, enhance, and/or prolong orgasmic sensation.

2. The device of claim 1, further configured to deliver said transcutaneous impulses a neuromuscular junction of a nerve innervating the bulbocavernosus muscle.

3-8. (canceled)

9. The device of claim 1, wherein one of the pads is configured to be entirely in contact with the perinium of the subject.

10-22. (canceled)

23. The device of claim 1, further configured for calibration comprising setting an intensity of said impulses to a muscle contraction threshold intensity.

24. A device for enhancement of a male orgasm, comprising
two or more muscle stimulation pads configured to attach to two or more of the following skin surfaces of a subject:
a perineum;
an abdomen; and
a lower back
one or more electrodes, in each of said muscle stimulation pads, configured to deliver transcutaneous electrical or magnetic impulses, wherein
said transcutaneous impulses are delivered to one or more of
a neuromuscular junction of a nerve innervating the bulbocavernosus muscle;
nerves innervating the prostate;
nerves innervating the seminal vesicles; and
said transcutaneously delivered impulses are configured to intensify, enhance, and/or prolong orgasmic sensation.

25. A method for enhancement of a male orgasm, comprising
attaching a plurality of muscle stimulation pads of the device of claim 1 to a perineum of a subject and at least one of
an abdomen of said subject; and
a lower back of said subject;
delivering electrical or magnetic impulses, by said device, to one or more of
nerves innervating the prostate; and
nerves innervating the seminal vesicles, wherein
said transcutaneously delivered impulses are configured to intensify, enhance, and/or prolong orgasmic sensation.

26. The method of claim 25, further comprising attaching a said muscle simulation pad to one or more of an abdomen and a lower back of said subject.

27. The method of claim 25, wherein the nerve innervating the bulbocavernosus muscle comprises a motor branch of the pudendal nerve.

28. The method of claim 25, wherein the nerves innervating the prostate comprise pelvic nerves and hypogastric nerves.

29. The method of claim 25, wherein the nerves innervating the seminal vesicles comprise hypogastric nerves and an inferior hypogastric plexus.

30. The method of claim 25, further comprising said muscle stimulation pad being entirely in contact with the perinium of the subject by.

31. The method of claim 25, further comprising inducing a continuous contraction of the bulbcavernosus muscle of the subject, by said transcutaneously delivered electrical or magnetic impulses.

32. The method of claim 25, further comprising enhancing said male orgasm by increasing contractility of the smooth muscles surrounding at least one of the prostate and the seminal vesicles, by said transcutaneously delivered electrical or magnetic impulses.

33. The method of claim 25, further comprising enhancing said male orgasm by increasing the fluid volume secreted by the prostate and/or seminal vesicles, by said transcutaneously delivered electrical or magnetic impulses.

34-35. (canceled)

36. The method of claim 25, further comprising generating increased intraurethral pressure and/or stronger bulbospongiosus muscle contractions of the subject by said transcutaneously delivered impulses.

37. The method of claim 36, further comprising generating greater fructose availability in tastebud-like structures in a urethra of the subject by said transcutaneously delivered impulses.

38. The method of claim 37, further comprising generating stronger neural signaling to higher neural centers.

39. The method of claim 38, further comprising inducing a more intense orgasm sensation for the subject by said transcutaneously delivered impulses.

40-41. (canceled)

42. The method of claim 25, wherein said impulses from said muscle simulation pads are delivered simultaneously.

43-46. (canceled)
